(19) United States
(12) Patent Application Publication
(10) Pub. No.: US 2021/0049533 A1
(43) Pub. Date: Feb. 18, 2021

(54) EXPIRATION MONITORING CONTROL MODULE

(71) Applicants: Bhuren Patel, Needham, MA (US); Parshavi Patel, Needham, MA (US)

(72) Inventors: Bhuren Patel, Needham, MA (US); Parshavi Patel, Needham, MA (US)

(21) Appl. No.: 16/884,496

(22) Filed: May 27, 2020

Related U.S. Application Data
(63) Continuation of application No. 15/968,031, filed on May 1, 2018, now abandoned.
(60) Provisional application No. 62/492,453, filed on May 1, 2017.

(51) Int. Cl. G06Q 10/06; G06Q 10/08; G06F 9/54

(52) U.S. Cl. CPC G06Q 10/06315; G06Q 10/087; G01N 33/02; G06Q 10/06314; G06F 9/542

(57) ABSTRACT

The present invention relates to novel methods, tools and systems that offer alternatives for more efficiently tracking products and monitoring the expiration status of goods with definable expiration. In particular, the tools and systems are capable of utilization by consumers, e.g., using consumer facing interfaces.

### RELATED APPLICATIONS

This application claims priority to co-pending U.S. Utility application Ser. No. 15/968,031, filed May 1, 2018, under Attorney Docket No. MYM-002, and which claims the benefit of priority of U.S. Provisional Patent Application No. 62/492,453, filed May 1, 2017, under Attorney Docket No. MYM-002-1; the entirety of each of which is incorporated herein by reference.

### BACKGROUND OF THE INVENTION

Sellers of goods with definable expiration, are plagued by the never-ending pursuit of monitoring expiration status of products. Such industries, including food stores, pharmacies, and pharmaceutical companies/manufacturers spend countless hours and money to maintain product quality and effectiveness for consumer purchase.

Manual methods of tracking the existence and monitoring the expiration status have attempted to account for such goods using the standard of “first in, first out.” Often professionals dedicated to this task are found to be less than 100% successful in these duties, resulting in unsafe food and drugs entering the market for delivery to consumers. Alternatively, certain radio frequency strategies have been employed for tracking purposes, but have found only limited industrial use.

Even further, there are currently no means of tracking and monitoring expiration of such products by consumers once purchase transfers ownership of these goods from seller to consumer. Very often the consumer faces the more difficult task of monitoring expiration dates, or just fails to do so given the amount of required effort. As such, the consumer typically finds out only when at the time the product is needed that such product has already expired.

Accordingly, there remains significant need for additional tools and systems that address these concerns and offer alternatives for more efficiently tracking products and monitoring the expiration status of goods with definable expiration; and in particular, tools and systems that are capable of utilization by consumers.

### SUMMARY OF THE INVENTION

Accordingly, the present invention is directed to novel methods, tools and systems that offer alternatives for more efficiently tracking products and monitoring the expiration status of goods with definable expiration. In particular, the tools and systems are capable of utilization by consumers, e.g., using consumer facing interfaces.

As such, one aspect of the present invention provides an expiration monitoring control (EMC) module capable of capturing expiration data, storing expiration data, analyzing expiration data to determine expiration status of a product, and generating action based on the expiration status of the product.

Another aspect of the present invention provides an expiration monitoring control system comprising an expiration monitoring control (EMC) module of present invention, capable of capturing expiration data, storing expiration data, analyzing expiration data to determine expiration status of a product, and generating action based on the expiration status of the product; and a device to capture expiration data of the product.

In another aspect, the present invention provides a method of expiration monitoring control comprising capturing expiration data, storing expiration data, analyzing expiration data to determine expiration status of a product, and generating action based on the expiration status of the product.

### BRIEF DESCRIPTION OF THE DRAWINGS

Advantages of the present apparatus will be apparent from the following detailed description, which description should be considered in combination with the accompanying drawings, which are not intended limit the scope of the invention in any way.

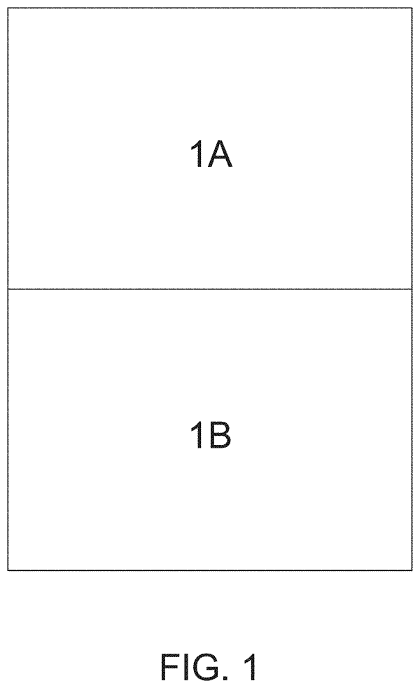

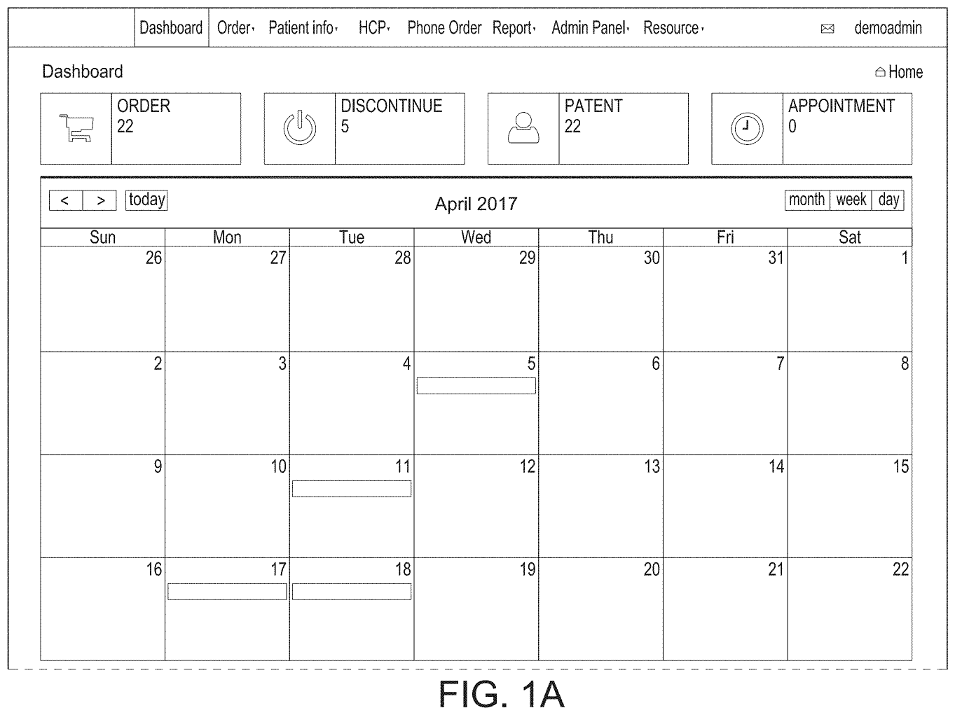

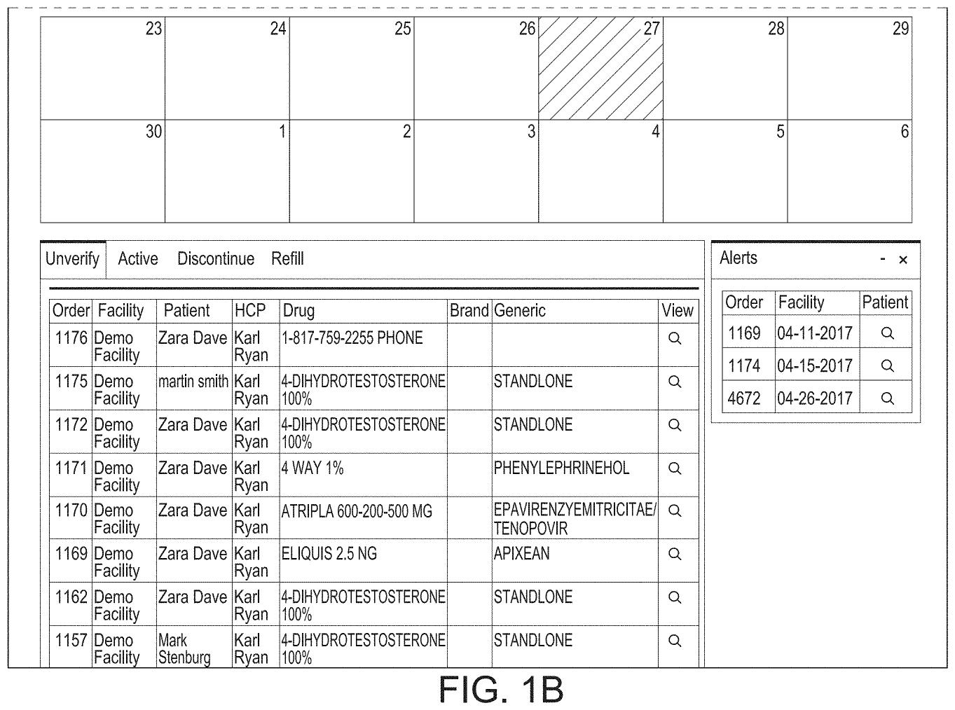

The attached FIG. 1, shown by the relation of FIG. 1A to FIG. 1B, and the combination of FIG. 1A with FIG. 1B depicts an example of the notification action as an alert on an EMC module consumer facing interface (lower right side) for certain particular embodiments of the interfaces of the present invention for the expiration monitoring control (EMC) modules described herein.

### DETAILED DESCRIPTION OF THE INVENTION

For years, monitoring the expiration of products or goods that have well-characterized expiration dates has been a manually intensive process. In contrast, however, the present invention is directed to novel methods, tools and systems that offer alternatives for more efficiently tracking products and monitoring the expiration status of goods with definable expiration. In particular, the tools and systems are capable of utilization by consumers, e.g., using consumer facing interfaces. Aside from the clear utility advantage the present invention provides, the tools, or modules, of the present invention provide both time and cost savings, as well as ensuring safe, unexpired, medical products are available on hand at all times, e.g., with products that are used infrequently and in an emergency, e.g., epinephrine.

The present invention, including expiration monitoring control (EMC) modules, interfaces, related methods, and systems thereof will be described with reference to the following definitions that, for convenience, are set forth below. Unless otherwise specified, the below terms used herein are defined as follows:

### 1. Definitions

As used herein, the term “a,” “an,” “the” and similar terms used in the context of the present invention (especially in the context of the claims) are to be construed to cover both the singular and plural unless otherwise indicated herein or clearly contradicted by the context.

The term “automatically” is used herein to describe a process that is automated or semi-automated. Automated processes do not contain steps that require a human operator to perform any steps. Semi-automated processes contain one or more steps that require a human operator; however, differ from manual processes by containing at least one step that does not require a human operator.

The language “consumer facing (CF) interface” is a user interface that is designed for use by a consumer of goods. In particular, such interface is designed for use by the consumer of the goods as opposed to the seller goods that would use this interface to monitor product for sale. More specifically, it is for use by the end purchaser, or agents thereof, e.g., caretakers or medical practitioners. As such, in certain embodiments of the invention, the interface is consumer facing only.

The term “expiration” is used herein to describe a date with which a product is labeled (or implicitly understood to be characteristic of such product) that beyond which the product has exceeded the anticipated shelf life. In certain embodiments, this date represents the date after which the product should no longer be used, either by operation of law or well-known convention. In particular embodiments, the expiration date is a date up until which the manufacturer guarantees the full potency and safety of the product. In specific embodiments, this date is absolutely definable, or definite, and is described herein as a definable expiration date.

The language “expiration data” is used herein to describe a combination of expiration date and product identification information. For clarity, expiration date would be considered a component of expiration data.

The term “HIPAA” is art-recognized, and is used herein as an abbreviation for the federal Health Insurance Portability and Accountability Act of 1996, a statute whose primary goal is to make it easier for people to keep health insurance, protect the confidentiality and security of healthcare information, and help the healthcare industry control administrative costs. In particular embodiments of the present invention, the interface is HIPAA compliant with respect to its ability to maintain the confidentiality and security of healthcare information.

The term “interface” is art-recognized, and is used herein to describe a shared boundary across which two or more separate interfacing components are enabled to exchange information, which can be, for example, between: software components, computer hardware components, peripheral device components, humans and combinations of thereof. Moreover, the operation of these separate components across the boundary, as in the interaction of a user, e.g., consumer, with the expiration monitoring control module and any related storage machine readable media, is referred to herein as “interfacing.” In particular embodiments, such interfacing may include accessing captured expiration information and related product information. In certain embodiments, the interfacing may be bi-directional. In other embodiments, the interfacing may be uni-directional. In specific embodiments, the term “interface” may be a user interface, e.g., a graphic user interface.

The language “machine-readable medium” is art-recognized, and describes a medium capable of storing data in a format readable by a mechanical device (rather than by a human). Examples of machine-readable media include magnetic media such as magnetic disks, cards, tapes, and drums, punched cards and paper tapes, optical disks, barcodes, magnetic ink characters, and solid state devices such as flash-based, SSD, etc. Machine-readable medium of the present invention are non-transitory, and therefore do not include signals per se, i.e., are directed only to hardware storage medium. Common machine-readable technologies include magnetic recording, processing waveforms, and barcodes. In particular embodiments, the machine-readable device is a solid state device. Optical character recognition (OCR) can be used to enable machines to read information available to humans. Any information retrievable by any form of energy can be machine-readable. Moreover, any data stored on a machine-readable medium may be transferred by streaming over a network. In a particular embodiment, the machine-readable medium is a network server disk, e.g., an internet server disk, e.g., a disk array. In specific embodiments, the machine-readable medium is more than one network server disks.

The language “notification action” is used herein to describe an action taken (such as an interface alert, email, text message, or the like) that notifies one or more groups of users, e.g., consumer or seller of the goods, upon the occurrence of a certain event. In the present invention, the notification is based on the expiration differential indicating the expiration of a product.

The term “product,” as used herein refers to a good that has definable and/or well-characterized expiration. Such products include goods selected from food product, medical products (e.g., drugs or devices), and cosmetic products, where the term “medical product” is used herein to describe any medicinal product or good with a well-characterized expiration date, e.g., medicines (such as pills, ointments, syringes, etc.) as well as devices. The term “food product” and “cosmetic product” are art-recognized, and coincides with the FDA definitions of these terms.

The term “storing” is art-recognized, and is used herein to describe the act of saving data on a machine readable medium in a manner that such data is subsequently retrievable on that machine readable medium.

### II. Methods of Expiration Monitoring Control of the Invention

Accordingly, the present invention provides a method of expiration monitoring control comprising capturing expiration data, storing expiration data, analyzing expiration data to determine expiration status of a product, and generating action based on the expiration status of the product, e.g., generating action based on the identification of the expiration of the product.

In certain embodiments, the method comprises the steps of:

capturing the expiration data of a product;

storing the captured expiration data of the medical product (e.g., may be stored on a second machine readable medium);

analyzing said stored expiration data of the product to determine an expiration status of the product, which is established from the expiration differential produced by comparison of the current date to the expiration date of the product; and

generating a notification action based on the expiration differential indicating the expiration of a product, e.g., within a defined period of time,

such that expiration of the product is monitored.

In certain embodiments of the methods of the invention, capturing the expiration data of a product may occur through manual data entry, scanning of product codes, e.g., UPC, or scanning image/text conversion.

In certain embodiments of the methods of the invention, the product is a medical product.

In certain embodiments of the methods of the invention, the product is a food product.

In certain embodiments of the methods of the invention, the notification action is an alert on an EMC module interface, e.g., on the dashboard.

In certain embodiments of the methods of the invention, the notification action is an email.

In certain embodiments of the methods of the invention, the notification action is a text message.

In certain embodiments of the methods of the invention, the notification action generates a reorder request, e.g., automatically reorders.

In certain embodiments of the methods of the invention, the interface presents a calendar of expiration dates for each product, e.g., on the dashboard.

In certain embodiments of the methods of the invention, the interface affords the ability to reorder the product.

In certain embodiments of the methods of the invention, the method further comprises the step of tracking the product. In particular embodiments, the step of tracking the product further comprises the step of storing product status (e.g., expiration status), count, and location. In particular embodiments, tracking information may additionally include information related to the product and/or the consumer.

In certain embodiments, the methods of the invention are not used in a unification tool for medication administration.

In certain embodiments, the methods of the invention are not used as an inventory tracking system for seller or manufacturer of goods.

### III. Expiration Monitoring Control Module of the Invention

The methods of the present invention are useful as instructions stored on a machine-readable medium for execution by a processor to perform the method. In certain embodiments, the methods and tools of the present invention also make use and/or comprise a processor. Accordingly, any methods of the present invention, alone or in combination with other methods (such as those described herein or elsewhere) may be stored on a machine-readable medium for execution by a processor to perform the method. Such a composition comprises an expiration monitoring control (EMC) module of the invention, e.g., an EMC module interface.

In this respect, the methods of expiration monitoring control provide for capturing expiration data, storing expiration data, analyzing expiration data to determine expiration status of a product, and generating action based on the expiration status of the product. In certain embodiments, the methods provide for interfacing with one or more users through an interfacing tool, e.g., on a server. The interfacing tools of the present invention are user interfaces for providing expiration monitoring control, e.g., graphical user interfaces (GUI), which are user-facing (e.g., consumer facing) for interacting with a user to transmit information, collect information, as well as act on that information through combination, analysis, and ultimately providing diagnostic analysis through expiration monitoring control.

One embodiment of the invention provides an expiration monitoring control (EMC) module capable of capturing expiration data, storing expiration data, analyzing expiration data to determine expiration status of a product, and generating action based on the expiration status of the product.

In certain embodiments, the EMC module comprises instructions stored on the machine readable medium for execution by a processor to perform a method comprising the steps of:

capturing the expiration data of a product;

storing the captured expiration data of the product (e.g., on a machine readable medium, e.g., may be stored on a second machine readable medium);

analyzing said stored expiration data of the product to determine an expiration status of the product, which is established from the expiration differential produced by comparison of the current date to the expiration date of the product; and

generating a notification action based on the expiration differential indicating the expiration of a product, e.g., within a defined period of time, such that expiration of the product is monitored.

In certain embodiments, the EMC module further comprises an EMC module interface.

In certain embodiments, the EMC module further comprises an EMC module interface, wherein the interface is a consumer facing (CF) interface.

In certain embodiments of the EMC module, the product is a medical product.

In certain embodiments of the EMC module, the product is a food product.

In certain embodiments of the EMC module, the notification action is an alert on the EMC module interface, e.g., on the dashboard.

In certain embodiments of the EMC module, the notification action is an email.

In certain embodiments of the EMC module, the notification action is a text message.

In certain embodiments of the EMC module, the notification action generates a reorder request, e.g., automatically reorders.

In certain embodiments of the EMC module, capturing the expiration data of a product may occur through manual data entry, scanning of product codes, e.g., UPC, or scanning image/text conversion.

In certain embodiments of the EMC module, the interface presents a calendar of expiration dates for each product, e.g., on the dashboard.

In certain embodiments of the EMC module, the interface affords the ability to reorder the product.

In certain embodiments of the EMC module, the method further comprises the step of tracking the product. In particular embodiments, the step of tracking the product further comprises the step of storing product status (e.g., expiration status), count, and location. In particular embodiments, tracking information may additionally include information related to the product and/or the consumer.

In certain embodiments of the EMC module, the machine-readable medium is selected from the group consisting of magnetic media, punched cards, paper tapes, optical disks, barcodes, magnetic ink characters, and solid state devices.

In certain embodiments, the EMC module is not used in a unification tool for medication administration.

In certain embodiments, the EMC module is not used as an inventory tracking system for seller or manufacturer of a goods.

### IV. Systems of the Invention

The expiration monitoring control (EMC) module of the present invention, described herein, may be incorporated into systems, which may also comprise one or more devices.

As such, another embodiment of the present invention provides an expiration monitoring control system comprising

an expiration monitoring control (EMC) module described herein, capable of capturing expiration data, storing expiration data, analyzing expiration data to determine expiration status of a product, and generating action based on the expiration status of the product; and

a device to capture expiration data of a product.

In certain embodiments of the invention, the system further comprises a display device for presenting an interface.

### A. Device to Capture Expiration Data

In certain embodiments, the systems of the present invention comprise a device to capture the expiration data of a product. The device affords the ability to semi-automate or automate certain data entry aspects of the expiration monitoring control (EMC) module, by capturing the expiration data of a product by scanning of product codes, e.g., UPC, or by scanning image/text conversion. Such devices include scanning devices with a primary utility of scanning information through image capture or text capture (e.g., including bar codes) that may or may not be operationally associated with a second device relied upon for interpretation of the image or text, or a device that has a secondary purpose that is capable of capturing image and text, e.g., wireless devices such as smartphones and tablets.

### B. Display Device for Presenting an Interface

In certain embodiments, the systems of the present invention comprise a display devices that are suitable for presenting an interface described herein comprising one or more wireless devices (e.g., smartphones, tablets, and laptop computers), desktop computers, or sufficiently operationally associated monitors, which are capable of interfacing with said expiration monitoring control (EMC) module.

### C. Additional Embodiments

In certain embodiments of the invention, the EMC module serves as a component of a system suitable for HIPAA compliance, through the creation and storage an electronic record that serves as an electronic medical record (EMR).

In certain embodiments of the invention, the EMC module serves as a component of a system suitable for use as a membership reward program. In particular embodiments, a member of the membership reward program may subscribe to the service providing expiration monitoring control through an EMC module, e.g., EMC module interface.

In certain embodiments of the invention, the EMC module serves as a component of a system suitable for use in an in-store product pickup program. In certain embodiments, the store and the consumer is alerted when a product expires, e.g., for the purposes of reorder (e.g., online reorder). In particular, the capturing of the expiration data is performed by the store before pick-up and posted to an account of the consumer.

In certain embodiments of the invention, the EMC module serves as a component of a system suitable for use in store loyalty programs. In certain embodiments, the store and the consumer is alerted when a product expires, e.g., for the purposes of reorder (e.g., online reorder). In particular embodiments, a mobile phone interface is provided by the store and relates to a consumer account. In particular embodiments, a scanning device is provided by the store, e.g., mounted to the carriages, and relate to a consumer account.

In certain embodiments of the invention, wherein the EMC module may be used through an interface, such as a graphic user interface, advertisement is used as a commercialization model. In particular embodiments, advertisement in a graphic user interface may be used as a revenue producing model (e.g., sales of advertising space, or advertising one's own goods).

### V. Design Aspects of the Invention

Independent of the utility related to interface of the expiration monitoring control (EMC) module of the present invention, the ornamental appearance of any novel design provided herein is intended to be part of this invention, for example, the interface design in the attached FIGURE, which may form an independent or combined ornamental appearance of the interface described herein.

Accordingly, one embodiment of the present invention provide an ornamental design for an interface of the expiration monitoring control (EMC) module as shown and described.

### EXEMPLIFICATION

Having thus described the invention in general terms, reference will now be made to the accompanying drawings of exemplary embodiments, which are not necessarily drawn to scale, and which are not intended to be limiting in any way.

In this respect, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

The attached FIGURE depicts an image of an example of the notification action as an alert on an EMC module consumer facing interface (lower right side) for certain particular embodiments of the interfaces of the present invention for the expiration monitoring control (EMC) modules described herein. The particular alert depicted in the interface (as a component or module of another interface) corresponds to the notification action of the EMC module, and is presented with information related to order number, expiration date, and a link for viewing additional information. Additional information may be presented in the alert, or held in the material that is linked therewith. In particular embodiments, alerts may be color coded, e.g., for urgency.

### INCORPORATION BY REFERENCE

The entire contents of all patents, published patent applications and other references cited herein are hereby expressly incorporated herein in their entireties by reference.

### EQUIVALENTS

Those skilled in the art will recognize, or be able to ascertain using no more than routine experimentation, numerous equivalents to the specific procedures described herein. Such equivalents were considered to be within the scope of this invention and are covered by the following claims. Moreover, any numerical or alphabetical ranges provided herein are intended to include both the upper and lower value of those ranges. In addition, any listing or grouping is intended, at least in one embodiment, to represent a shorthand or convenient manner of listing independent embodiments; as such, each member of the list should be considered a separate embodiment.

What is claimed is:

1. An expiration monitoring control (EMC) module capable of capturing expiration data, storing expiration data, analyzing expiration data to determine expiration status of a product, and generating action based on the expiration status of the product.

2. The expiration monitoring control (EMC) module of claim 1, wherein the EMC module comprises instructions stored on the machine readable medium for execution by a processor to perform a method comprising the steps of:
capturing the expiration data of a product;
storing the captured expiration data of the product;
analyzing said stored expiration data of the product to determine an expiration status of the product, which is established from the expiration differential produced by comparison of the current date to the expiration date of the product; and
generating a notification action based on the expiration differential indicating the expiration of a product,
such that expiration of the product is monitored.

3. The expiration monitoring control (EMC) module of claim 1, further comprising an EMC module interface.

3. The expiration monitoring control (EMC) module of claim 1, wherein the interface is a consumer facing (CF) interface.

4. The expiration monitoring control (EMC) module of claim 1, wherein the product is a medical product.

5. The expiration monitoring control (EMC) module of claim 1, wherein the product is a food product.

6. The expiration monitoring control (EMC) module of claim 3, wherein the notification action is an alert on the EMC module interface.

7. The expiration monitoring control (EMC) module of claim 1, wherein the notification action is an email.

8. The expiration monitoring control (EMC) module of claim 1, wherein the notification action is a text message.

9. The expiration monitoring control (EMC) module of claim 1, wherein the notification action generates a reorder request.

10. The expiration monitoring control (EMC) module of claim 1, wherein capturing the expiration data of a product may occur through manual data entry, scanning of product codes, or scanning image/text conversion.

11. The expiration monitoring control (EMC) module of claim 3, wherein the interface presents a calendar of expiration dates for each product.

12. The expiration monitoring control (EMC) module of claim 3, wherein the interface affords the ability to reorder the product.

13. The expiration monitoring control (EMC) module of claim 2, wherein the method further comprises the step of tracking the product.

14. The expiration monitoring control (EMC) module of claim 13, wherein the step of tracking the product further comprises the step of storing product status, count, and location.

15. The expiration monitoring control (EMC) module of claim 1, wherein the machine-readable medium is selected from the group consisting of magnetic media, punched cards, paper tapes, optical disks, barcodes, magnetic ink characters, and solid state devices.

16. A expiration monitoring control system comprising
an expiration monitoring control (EMC) module of claim 1, capable of capturing expiration data, storing expiration data, analyzing expiration data to determine expiration status of a product, and generating action based on the expiration status of the product; and
a device to capture expiration data of the product.

17. The expiration monitoring control system of claim 16, further comprising a display device for presenting an interface.

18. A method of expiration monitoring control comprising capturing expiration data, storing expiration data, analyzing expiration data to determine expiration status of a product, and generating action based on the expiration status of the product.

19. The method of claim 18, wherein the method comprises the steps of:
capturing the expiration data of a product;
storing the captured expiration data of the medical product;
analyzing said stored expiration data of the product to determine an expiration status of the product, which is established from the expiration differential produced by comparison of the current date to the expiration date of the product; and
generating a notification action based on the expiration differential indicating the expiration of a product,
such that expiration of the product is monitored.

20. The method of claim 19, wherein capturing the expiration data of a product may occur through manual data entry, scanning of product codes, or scanning image/text conversion.
